(19) United States
(12) Patent Application Publication
(10) Pub. No.: US 2013/0108666 A1
(43) Pub. Date: May 2, 2013

(54) GLASS/CARBON NANOTUBE COMPOSITE MATERIAL FOR BONE GRAFT SUPPORT

(71) Applicant: Jing Zhang, Indianapolis, IN (US)

(72) Inventor: Jing Zhang, Indianapolis, IN (US)

(21) Appl. No.: 13/287,614

(22) Filed: Nov. 2, 2011

(51) Int. Cl. A61K 9/00; C03B 19/06; A61P 19/08; C03B 19/00; B82Y 5/00

(52) U.S. Cl. 424/400; 264/122; 264/434; 977/904

(57) ABSTRACT

A composition for bone graft structural support, including a bioglass matrix and a plurality of carbon nanotubes dispersed throughout the bioglass matrix. The carbon nanotubes are generally cylindrical and are substantially between about 10 nanometer and about 20 nanometers in diameter and are substantially between about 5 nanometers and about 13 nanometers in length.

### ACKNOWLEDGEMENT

Research leading to this novel technology was federally supported by grant no. 0723244 from the National Science Foundation. The United States government retains certain rights in this novel technology.

### TECHNICAL FIELD

The novel technology relates generally to materials science, and, more particularly, to a carbon nanotube/bioglass composite system.

### BACKGROUND

Ceramic based biomaterials are used in a wide range of human skeletal repair and restoration applications and can be used as a synthetic bone substitute. Powdered bioglass has been used as a bone replacement material for decades. Bioglass was originally developed as a bioactive material to aid in the repair of bone and tissue by forming a direct bond with the affected tissue. In addition to biocompatibility, glass-ceramics typically have relatively high mechanical strengths, low coefficients of thermal expansion, and good dielectric properties. Bioglass has a composition of about 24.5 wt % Na₂O, 24.5 wt % CaO, 45-wt % S2O₂, and 6-wt % P₂O₅ and has a density of about 2.70 g/cm³ with a softening point of around 1070° C. Bioglass glass powder is typically manufactured by reacting and fusing batch raw materials in a platinum crucible, quenching the mixture, crushing the result, and then grinding and sieving the material to obtain appropriately sized particles. Bioglass powder has been used successfully as bone-filling material in orthopedic and dental surgeries, but its lean mechanical strength limits its applications in load-bearing positions. In order to bolster the mechanical properties of bioglass, it is often reinforced with other materials such as metals, polymers, and ceramics.

### SUMMARY

The present novel technology relates to bioglass/carbon nanotube composite materials.

One object of the present novel technology is to provide an improved bioglass material for in vitro utilization. Related objects and advantages of the present novel technology will be apparent from the following description.

### BRIEF DESCRIPTION OF THE DRAWINGS

FIG. 1A is an SEM image of the bioglass powder precursor component of the present composite material.

FIG. 1B graphically is a TEM image of the multi-wall carbon nanotube component of the composite material of the present novel technology.

FIG. 2A graphically shows the flexural strength of the bioglass/carbon nanotube composite as a function of carbon nanotube content.

FIG. 2B graphically shows the fracture toughness of the bioglass/carbon nanotube composite as a function of carbon nanotube content.

FIG. 2C graphically shows the hardness of the bioglass/carbon nanotube composite as a function of carbon nanotube content.

FIG. 3A is a photomicrograph of the fracture surface of a composite material having 5 weight percent carbon nanotubes dispersed in a bioglass matrix and densified with the SPS technique.

FIG. 3B is a photomicrograph of the fracture surface of a composite material having 9 weight percent carbon nanotubes dispersed in a bioglass matrix and densified with the SPS technique.

FIG. 4 is a photomicrograph of an indentation crack propagating through a composite having 5wt. % multi-walled carbon nanotubes dispersed in a bioglass matrix, illustrating the bonding of the nanotubes and the pullout mechanism, and showing bridging nanotubes evident along the crack.

### DESCRIPTION OF THE PREFERRED EMBODIMENTS

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated device and such further applications of the principles of the novel technology as illustrated therein being contemplated as would normally occur to one skilled in the art to which the novel technology relates.

Carbon nanotubes (CNTs) have evoked tremendous interest since their discovery in 1991because of their extraordinary intrinsic mechanical, electrical, and thermal properties as well as their high aspect ratios. The impressive mechanical properties of nanotubes make them an attractive fiber choice for many ceramic composites and are attributed to their unique structure. Nanotubes can have strength of around 150 GPa or more and Young's modulus of about 1200 GPa, far exceeding the strength and modulus of steel (0.4 GPa and 208 GPa, respectively). These strength properties are a consequence of the nanotube structure, specifically the high aspect or surface-to-volume ratio, length to width parameter, and the covalent sp2 bonds formed between the individual carbon atoms

The present novel technology relates to carbon nanotube 10 reinforced bioactive glass matrix 20 (or bioglass) composites 30. The novel composites may be formed by sintering techniques, such as spark plasma sintering (SPS) and conventional compaction and sintering. The composites 30 show improved mechanical properties. Using SPS, a bioglass 20/carbon nanotube 10 composite 30 may be formed having increased flexural strength and fracture toughness, 159% and 105% that of unreinforced bioglass, respectively. Enhanced strength and toughness mechanisms are attributed to the interfacial bonding 40 and bridging effects between the carbon nanotubes 10 and bioglass matrix 20 during crack propagations.

The composites 30 were densified without damaging the carbon nanotubes 10 during sintering by two sintering techniques, spark plasma sintering (SPS) and conventional sintering. SPS is a moderate pressure sintering method based on the conjecture of a high-temperature plasma momentarily generated in the gaps between powder materials by electrical discharge during direct current (DC) pulsing. The SPS process has a number of advantages over conventional sintering methods, such as hot pressing. Materials can be sintered using SPS in a matter of minutes as opposed to hours. Further, the required temperatures needed to consolidate a compact 30 to full density are typically significantly lower.

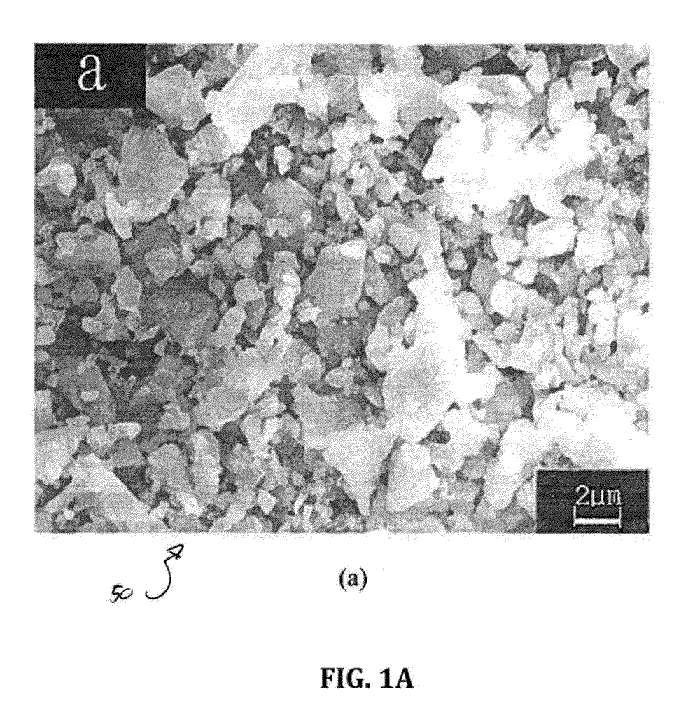

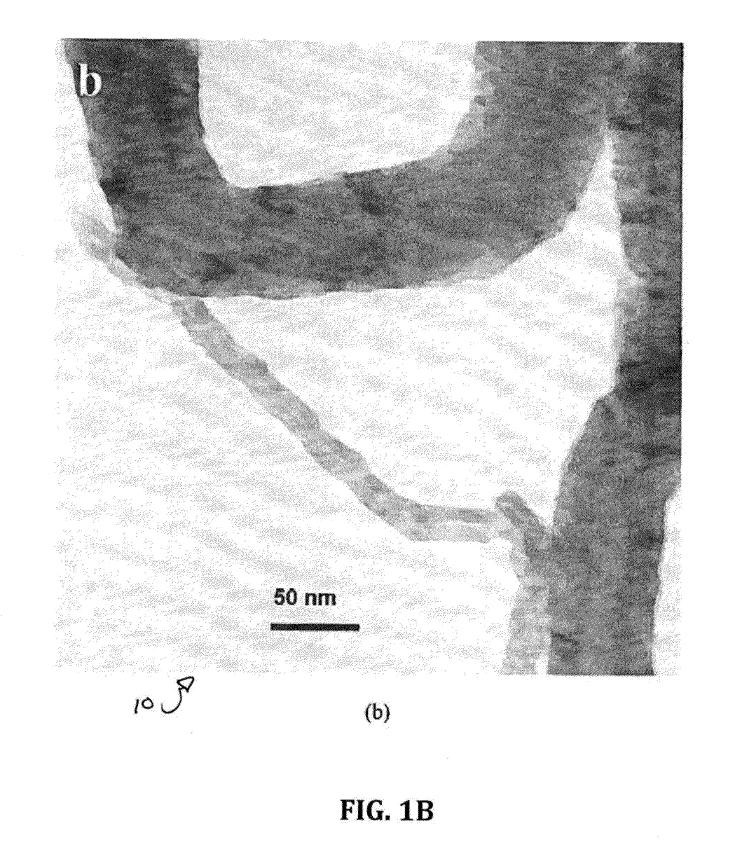

Bioglass powder 50 was mixed with different weight fractions of multi-walled carbon nanotubes 10 (1 wt. %, 3 wt. %, 5 wt. %. 7 wt. % and 9 wt. %, respectively, although other higher or lower weight fractions or concentrations may be selected). The starting materials were bioglass powders 50 and multi-wall carbon nanotubes 10 (or MWCNTs). The bioglass powder 50 had a chemical composition in weight percent of: 45% SiO₂; 24.5% Na₂O; 24.5% CaO and 6% P₂O₅. The average size of powders was less than 10 mm and the purity was about 99.6%. The carbon nanotubes 10 were typically generally cylindrical and are typically between about 2 μm and about 50 μm in diameter and more typically between about 10 μm and about 20 μm in diameter, and typically between about 2 μm and about 15 μm in length and more typically between about 5 μm and about 13 μm in length, although the nanotubes 10 may be longer or shorter and/or wider or narrower, as desired. The purity of the carbon nanotubes was 99%. FIG. 1A is an SEM photomicrograph of typically bioglass powder 50 and FIG. 1B is a TEM image of the multi-walled carbon nanotubes 10.

The composite 30 was fabricated by mixing the carbon nanotubes 10 and glass powder 30 to yield various admixtures with the respective above-listed compositions, forming the admixtures into green bodies or compacts, and sintering the compacts to yield densified bodies 30. In general, the compacts were densified by heating them to a temperature above the glass transition temperature but below the softening point, such that viscous deformation is possible. Densification can be achieved pressurelesly or through the application of pressure, such as by hot pressing. Typically, densification is achieved under an inert or reducing atmosphere, or in a partial vacuum, in order to minimize oxidation of the carbon nanotubes at elevated temperatures. For SPS sintering, the various compositions may be mixed by wet or dry methods, such as by using a high energy ball milling machine. Alcohol typically may be used as dispersant media to prevent severe agglomeration of the powders in the wet mill.

For a composite 30 formed through conventional sintering or densification techniques, a ball mill was used with a 250 ml zirconium oxide grinding bowl and 1 mm steatite balls to mix the bioglass powder and the carbon nanotubes. Low-density steatite balls were used so as to minimize damage to the carbon nanotubes and milling of the bioglass during mixing. The raw materials were measured into the zirconium bowl along with polyvinyl alcohol (1 ml 10 wt %), added to the admixture to function as a binder to improve the green strength during compaction. In order to minimize milling of the bioglass particles 50 and to separate carbon nanotube 10 clusters, a high-energy mix (here, 300 rpm for a period of 10 minutes) was utilized to break apart the agglomerated nanotube clusters. Following the high-energy mix, the material was mixed at 100 rpm for a period of 50 minutes. A ball-to-powder ratio of 5:1 was used for both mixing processes. The product was then separated from the mixing balls and containerized.

For the composite densified through the SPS technique, the mixed powders were filled into a mold and then pressed to produce a composite by SPS process. The powders were loaded into a graphite die having a 30 mm inner-diameter. Graphitic paper was placed between the punch and the powders as well as between the die and the powders for easy removal. The powders were sintered in a vacuum (less than 5 Pa). The heating rate was 100° C./min with a sintering temperature setpoint of 850° C., the pressure was 40 MPa, and sintering conditions were held for 10 minutes. For comparison, a pure bioglass specimen was also prepared by the same method.

For conventional sintering, each 3 gram composite sample was cold pressed in a 19.3 mm diameter steel die using a hydraulic press. Pressure on the die was increased to 11 MPa and held for a period of 5 minutes and then increased to 33 MPa for 55 minutes to compact the sample. After compaction, average sample thickness was measured to be 10.6 mm with a diameter of 19.3 mm. An electrical heat treatment furnace with a 3.3 m³ controlled atmosphere chamber was used to sinter the composites. The composites were sintered at 850° C. The samples were positioned onto a firebrick and into the furnace at room temperature. Upon initiation of the electrical heating element the chamber was purged with argon for 15 minutes. In order to ensure a 100% argon environment, a flow rate of 6.91 CFH, equivalent to 20.3 chamber changes per hour, was directed through the furnace chamber during heating, sintering, and cooling of the samples. Once the argon environment was established, the samples were heated to 850° C. and sintered for 20 minutes. This time/temperature profile developed to eliminate sample porosity, achieve bioglass crystallization, preserve the multi-walled carbon nanotubes, and encourage interfacial bonding of the bioglass matrix and carbon nanotubes.

The microstructure characterization was carried out on a field emission scanning electron micrograph (FESEM) and an optical microscope. Densities of the consolidated composite specimens were obtained using Archimedes' method with distilled water as the intrusion medium. The theoretical densities of bioglass (2.80 g/cm³) and carbon nanotubes (1.75 g/cm³, provided by the manufacturer) were used to calculate the relative density of products. Flexural strength test specimens with the dimensions of 2.5 mm×5 mm×25 mm and hardness test specimens with the dimensions of 2.5 mm×10 mm×10 mm were cut from sintered disks using a diamond saw, and then the specimens were polished using standard metallographic procedures, utilizing SiC sandpaper (300, 400, and 600 grit), followed by progressively smaller diamond slurries of 9, 3. 1, and 0.25 mm diam. Between each step the samples were sonicated in water.

Three-point bending tests were conducted to evaluate the flexural strength. Flexural strength=3 FL/2 wh², and flexural modulus E=L³F/4 wh³γ, where F is the fracture load, L is the distance between the two outer points, w is the width of the specimen, h is the height of the specimen and γ is the deflection of the beam when a force F is applied. Indentation experiments were carried out to measure the hardness and fracture toughness of the synthesized products using a micron hardness tester: Hᵥ=1.82*107P/D²,K_{IC}=α(E/Hᵥ)^{1/2}(P/d^{3/2}), where Hᵥ is the Vickers hardness. P is the indention load in Newton, D the indentation diagonals in meter. K_{IC} the fracture toughness, α=0.016, a geometric parameter, 2d is the secondary crack length in meter and E is flexural modulus. At least five specimens were tested for each test condition.

Under the SPS conditions of sintering temperature 850° C., pressure 40 MPa and holding time 10 minutes, near fully dense composites (at least about 99 percent dense or having less than about 1 percent porosity) were achieved for the 1 wt. %, 3 wt. % and 5 wt. % compositions, as shown in Table 1. Compared with pure sintered bioglass, the hardness of the composites decreases with carbon nanotube concentration, but both the strength and toughness are improved substantially.

TABLE 1
The mechanical properties of the sintered MWCNT/45s5Bioglass
composites of SPS and conventional sintering (CS).

| | relative | flexural | | fracture |
|---|---|---|---|---|
| | density | strength | hardness | toughness |
| | (%) | (MPa) | (Hv) | (MPa m^{1/2}) |
| Materials | SPS/CS | SPS/CS | SPS/CS | SPS/CS |
| 45s5Bioglass | 100/83.5 | 41 ± 4/— | 620 ± 11/ | 0.57 ± 0.08/ |
| | | | 377 ± 55 | 0.38 ± 0.11 |
| 1 wt. % CNT/BG | 99/82.5 | 61 ± 6/— | 400 ± 7/ | 0.68 ± 0.10/ |
| | | | 359 ± 55 | 0.48 ± 0.23 |
| 3 wt. % CNT/BG | 100/82.9 | 86 ± 8/— | 379 ± 9/ | 0.92 ± 0.06/ |
| | | | 161 ± 53 | 0.21 ± 0.07 |
| 5 wt. % CNT/BG | 100/81.2 | 106 ± 8/— | 395 ± 6/ | 1.17 ± 0.11/ |
| | | | 146 ± 98 | 0.16 ± 0.06 |
| 7 wt. % CNT/BG | 96/71.6 | 100 ± 10/— | 335 ± 12/ | 0.75 ± 0.05/ |
| | | | 86 ± 35 | 0.15 ± 0.06 |
| 9 wt. % CNT/BG | 91/64.5 | 58 ± 9/— | 264 ± 10/ | 0.80 ± 0.09/ |
| | | | 30 ± 14 | 0.04 ± 0.01 |

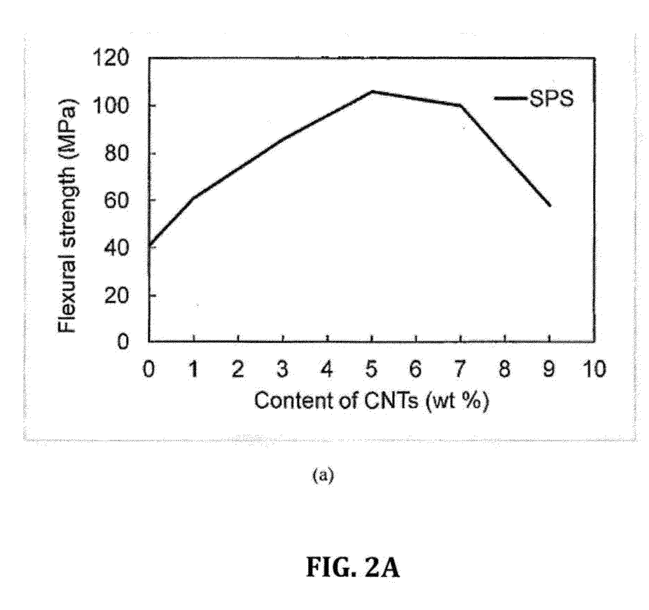

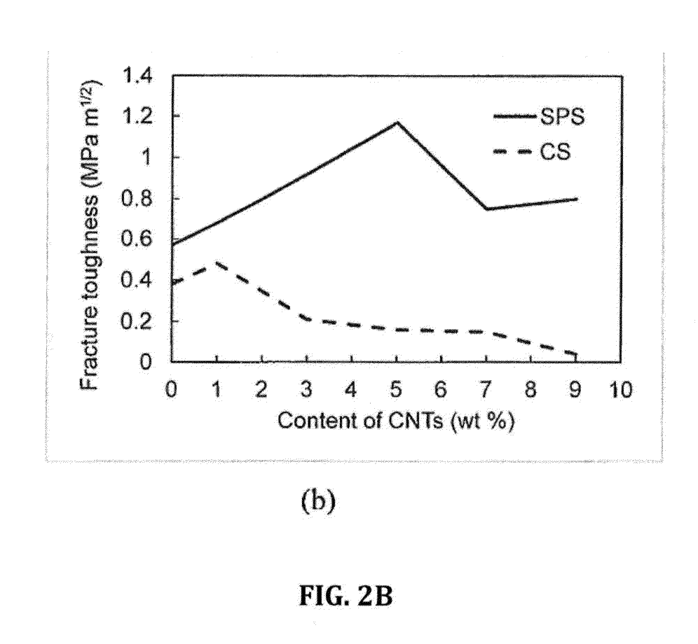

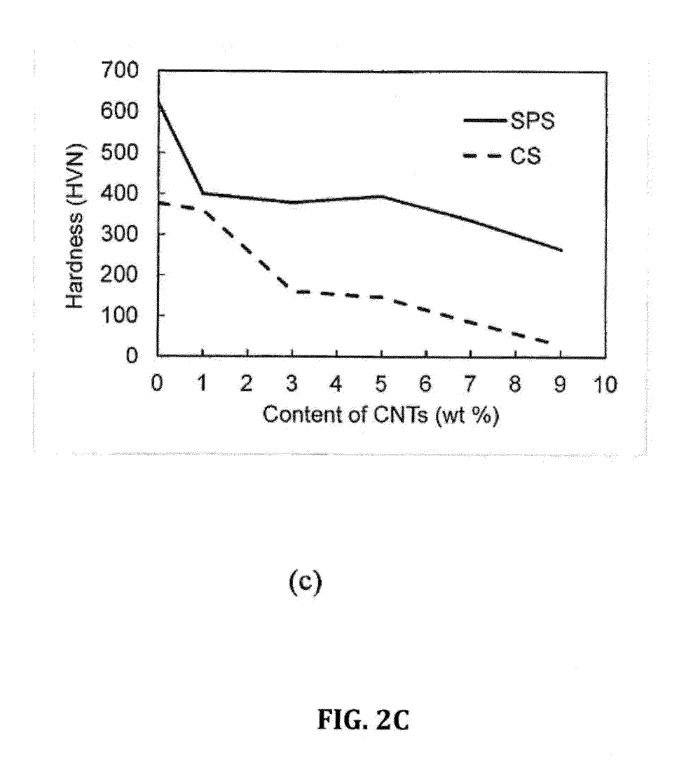

FIG. 2A illustrates the relationship between the flexural strength of composites 30 and the weight fraction or concentration of carbon nanotubes 10. Using SPS, the flexural strength of the composites 30 increases with the increase in weight fraction of multi-walled carbon nanotubes 10 from 1 to 9 wt. %. The addition of 5 wt. % multi-walled carbon nanotubes 10 increases the bioglass matrix 20 flexural strength from 41 to 106 MPa (a 159% increase). This is due to the good multi-walled carbon nanotube-bioglass interfacial bonding 40, with the multi-walled carbon nanotubes 10 being homogeneously dispersed well within the bioglass matrix 20. However, the strengthening effect of multi-walled carbon nanotubes 10 reduces with a further increase in the multi-walled carbon nanotube weight fraction to 9 wt. %, as the flexural strength decreases from 106 to 58 MPa. However, it is still an increase over the strength of the pure bioglass sintered reference standard part (41 MPa). The decrease is mainly attributed to the composites' lower relative density due to the agglomeration of additional multi-walled carbon nanotubes 10, which have a lower density than the glass matrix 20. The agglomeration of multi-walled carbon nanotubes 10 tends to weaken the bonding between the carbon nanotubes 10 and the bioglass matrix 20.

FIG. 2B illustrates the relationship between the fracture toughness of composites 30 and the weight fraction of multi-walled carbon nanotubes 10, as formed using SPS and conventional sintering. The fracture toughness of the composites 30 shows a similar trend as the flexural strength. The addition of 5 wt. % multi-walled carbon nanotubes 10 increases the bioglass matrix 20 fracture toughness from 0.57 to 1.17 MPa m^{1/2} (a 105% increase). This is likely due to the multi-walled carbon nanotubes 10 dispersion in the matrix, which serves as a reinforcing phase. For conventional sintering, the maximum fracture toughness occurs at a carbon nanotube 10 concentration of about 1 wt. %. Further addition of multi-walled carbon nanotubes 10 actually reduces the fracture toughness of the composites 30. The different relationships of SPS and conventional sintering may arise from differences in the mixing procedures, damage to the carbon nanotubes 10 upon heat treatment, and/or resultant relative density differences (Table 1).

FIG. 2C shows the effect of multi-walled carbon nanotubes 10 on the hardness of the composites 30. Both SPS and conventional sintering yield hardness decreases with an increase in the concentration of multi-walled carbon nanotubes 10. Hardness is typically related to the strength of the material, and the decrease in hardness is generally attributed to the addition of the relatively soft multi-walled carbon nanotubes 10 into the hard bioglass matrix 20.

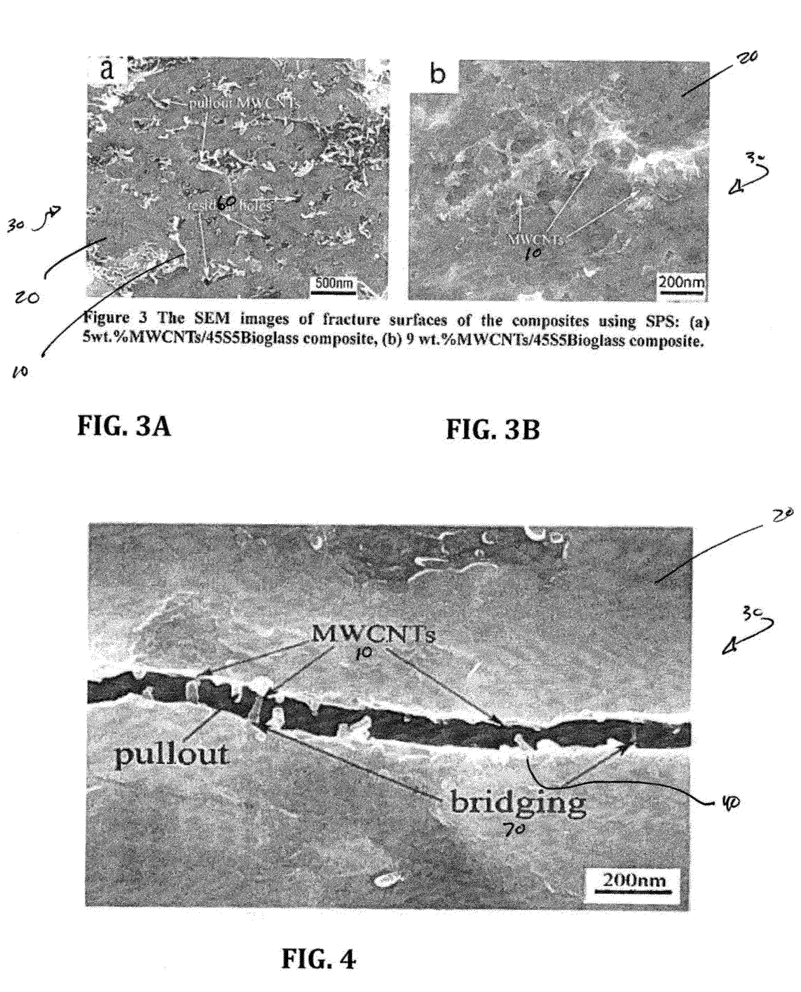

The fracture surfaces of the SPS composites 30 obtained after flexural strength tests are shown in FIG. 3. The multi-walled carbon nanotubes 10 are homogeneously dispersed within the bioglass matrix 20 in the 5 wt. % multi-walled carbon nanotube 10/bioglass matrix 20 (FIG. 3A) composites 30. There may be seen evidence of pullout of multi-walled carbon nanotubes 10, including residual holes 60 left by pulled out multi-walled carbon nanotubes 10, indicating that the presence of an ideal multi-walled carbon nanotube 10/bioglass matrix 20 interfacial structure is suitable for crack deflection via the pullout mechanism. This crack deflection mechanism likely results in the increase of fracture toughness, and, since the elastic modulus of multi-walled carbon nanotubes 10 is much higher than that of the bioglass matrix 20, the modulus-load-transfer also increases fracture toughness by transferring stresses at a crack tip to the regions remote from the crack tip, hence decreasing the stress intensity at the crack tip.

FIG. 3B shows the fracture surface of the composites with 9 wt. % multi-walled carbon nanotubes 10. The multi-walled carbon nanotubes 10 are relatively non-uniformly dispersed within the bioglass matrix 20, resulting in the lower relative density of the composites 30. Hence the flexural strength and fracture toughness lower than those with the weight fraction of 5 wt. %

FIG. 4 illustrates a typical SEM image of the crack propagation paths produced by Vickers indentation. It can be observed that the multi-walled carbon nanotubes 10 in the wake of crack propagation bridge 70 the two crack surfaces, which suggests operation of the crack bridging 70 effect during crack propagation, thus enhancing both the strength and the toughness of the composite.

Multi-wall carbon nanotube 10/bioglass matrix 20 composites 30 may be successfully synthesized by means of a mechanical alloying process followed by spark plasma sintering (SPS) technique and conventional sintering. The mechanical properties of the composites formed using SPS are typically superior to those formed by means of conventional sintering. The optimal concentration of nanotubes appears to be about 5 wt. % when densified through the SPS process. Compared with the pure bioglass matrix, the hardness of composites appreciably decrease with increasing concentration of carbon nanotubes 10 while the flexural strength and fracture toughness substantially increase with nanotube concentration over the observed concentration range, up to about 159% (up to 106 MPa) and 105% (up to 1.17 MPa^{1/2}), respectively. Enhanced strength and toughness mechanisms likely arise from the interfacial bonding 40 and bridging effects 70 between the carbon nanotubes 10 and bioglass matrix 20 during crack propagations. The composites 30 appear to have sufficient strength and toughness to perfume in orthopedic material applications.

In operation, the bioglass matrix 20/carbon nanotube 10 composites 30 may be implanted in vitro as synthetic bone graft materials for general orthopaedic, craniofacial, maxillofacial and periodontal repair, as cochlear implant materials, and as bone tissue engineering scaffolds. The composites 30 may be implanted without the need of, or with minimal need of, additional structural materials, such as ceramic, steel or titanium implants.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

I claim:

1. A composition for bone graft structural support, comprising:
a bioactive glass matrix; and
a plurality of carbon nanotubes dispersed throughout the bioactive glass matrix to define a composite material;
wherein the carbon nanotubes are generally cylindrical;
wherein the carbon nanotubes are substantially between about 10 nanometer and about 20 nanometers in diameter; and
wherein the carbon nanotubes are substantially between about 5 nanometers and about 13 nanometers in length.

2. The composition of claim 1 wherein the bioactive glass matrix is about 45 weight percent SiO₂, about 24.5 weight percent Na₂O, about 24.5 weight percent CaO, and about 6 weight percent P₂O₅.

3. The composition of claim 1 wherein the carbon nanotubes are at least about 99 percent pure.

4. The composition of claim 1 wherein the carbon nanotubes are bonded to the bioactive glass matrix.

5. The composition of claim 1 wherein the composite material is at least about 99 percent dense.

6. A method for making a composite material, comprising:
combining a quantity of powdered bioglass with a predetermined amount of carbon nanotubes to define an admixture;
homogenizing the admixture;
forming the homogenized admixture into a green body;
densifying the green body to yield a densified body defining a homogeneous dispersion of carbon nanotubes in a bioglass matrix.

7. The method of claim 6 and further comprising adding a quantity of binder to the admixture.

8. The method of claim 6 wherein densification occurs through pressureless sintering.

9. The method of claim 6 wherein densification occurs through spark plasma sintering of the green body.

10. The method of claim 6 wherein densification occurs through hot pressing.

11. The method of claim 6 wherein densification yields a densified body having less than about 1 percent porosity.

12. The method of claim 6 wherein the carbon nanotubes dispersed throughout the bioglass matrix are substantially undegraded by the densification process.

13. The method of claim 5 wherein the admixture is homogenized in a ball mill with spherical mixing media.

14. The method of claim 5 wherein the carbon nanotubes dispersed throughout the bioglass matrix are bonded to the bioglass matrix.
